(19) United States
(12) Patent Application Publication
(10) Pub. No.: US 2018/0116969 A1
(43) Pub. Date: May 3, 2018

(54) Multi-shell Capsule

(71) Applicant: Mikhail Kozyrev, Brandon, TX (US)

(72) Inventor: Mikhail Kozyrev, Brandon, TX (US)

(21) Appl. No.: 15/794,953

(22) Filed: Oct. 26, 2017

Related U.S. Application Data
(60) Provisional application No. 62/413,464, filed on Oct. 27, 2016.

(51) Int. Cl. A61K 9/48

(52) U.S. Cl. CPC A61K 9/4808; A61K 9/4891

(57) ABSTRACT

The invention provides a multi-shell capsule for oral administration of a combination of pharmaceutical and/or nutrient ingredients (hereinafter referred to as active ingredient) which release the active ingredient or set of active ingredients at different dissolvent time at a gastrointestinal tract at a different section of gastrointestinal tract (depending on dissolvent time).

### BACKGROUND

### 1. Field of the Invention

The present invention relates to multi-shell capsule for oral administration of a combination of the active ingredients. The capsule is formulated so that the shells release each active ingredient or set of ingredients at different dissolvent time or (depending on dissolvent time) at a different section of gastrointestinal tract.

### 2. Description of the Prior Art

Published U.S. Application No. 2008/0113026 discloses a layered active formulation including two or more active layers and an intermediate layer disposed between the two or more active layers. A formulation wherein the first active layer comprises phentermine and the second active layer comprises topiramate is disclosed.

A delayed/extended release formulation of topiramate for once-a-day administration is disclosed in published U.S. Patent Application No. 2008/0085306.

Published U.S. Patent Application No. 200610121112 also discloses a topiramate formulation with an immediate release component of topiramate and a delayed release component of top ira mate.

However, there is a problem of a stable, safe, oral active composition having therapeutic efficacy, in a single dose unit comprising drugs, dietary supplements, vitamins, essential nutrients, mineral nutrients, essential fatty acids, and essential amino acids physically or chemically incompatible with one another.

Accordingly, there is a need to apply active substances of medicines, vitamins, dietary supplements for a treatment or prophylaxis, some of these substances may be physically or chemically incompatible with one another or might require different body conditions to be metabolized in the body. In particularly, it is needed to take different substances at different dissolvent time.

### SUMMARY OF THE INVENTION

An aspect of the present invention relates to a multi-shell capsule comprising at least two active ingredients. The multi-shell capsule is formulated so that the different shells dissolve and release the active ingredients at different dissolvent time at a gastrointestinal tract or (depending on dissolvent time) at a different section of gastrointestinal tract.

In a preferred embodiment of this invention, the multi-shell capsule is used for dietary supplements, vitamins, essential nutrients, mineral nutrients, essential fatty acids, essential amino acids and oral medicines, where each shell or sub-capsule contains a single active ingredient or a plurality of active ingredients that is separated from each other for the purpose to dissolve the active ingredients at gastrointestinal tract at different dissolvent time. This provides the necessary spacing between the assimilation of different substances in the body.

In one preferred embodiment of this invention, each ingredient or set of ingredients packed to the multi-shell capsule can be dissolved, absorbed and digested at different times and in different parts of the gastrointestinal tract without interfering to each other.

In another preferred embodiment of this invention, the multi-shell capsule invention provides an effective way to compose different supplements in one pill or capsule with dissolve-timing control as an alternative to separating them into a plurality of doses which need to be taken at different time.

The multi-shell capsule is coated with a functional coating or non-functional coating around the entire surface of the capsule. In this embodiment, the capsule may further comprise an optional subcoating between the secondary shells and the functional coating or non-functional coating. The multi-shell capsule may further comprise optional inert layers to control release of the active ingredient from the secondary shell(s). By “inert layer” it is meant a layer containing no active ingredients or active ingredients in an amount which is insignificant to the therapeutic activity of the mini tablet or formulations thereof.

As will be understood by the skilled artisan upon reading this disclosure, additional primary shells and/or secondary shells containing one or more active ingredients and/or additional inert layers can be added to the capsule, and capsules comprising these additional layers are encompassed by the present invention. Addition of such layers in no way circumvents the present invention.

The secondary shell(s) of the multi-shell capsule are formulated, for example, by preparing a powder or granular mixture of drugs or nutrients with release retarding excipients and/or other acceptable active excipients by dry blending, slugging or dry or wet granulating, optionally adding a lubricant and compressing the mixture into capsule shells.

The optional inert layer or layers may comprise any biocompatible compound or mixture of compounds. The inert layer may be soluble or insoluble, permeable or impermeable, pH dependent or pH independent or any combination thereof depending upon the drug(s) or nutrient(s) to be orally administered and/or the release mechanism required. Preferably, the inert layers are inert, insoluble and impermeable to drug contained in the modified release layer(s). Accordingly, the inert layer preferably comprises no drug(s) or nutrient(s) in an amount which is insignificant to the therapeutic activity of the multi-shell capsule or formulations thereof.

Furthermore, one or more of the primary shell(s) and one or more of the secondary shells and optionally, one or more of the inert layers are then compressed together to form a single multi-shell capsule of the present invention.

Each layer containing one ingredient or set of active ingredients dissolves in the gastrointestinal tract at a different dissolvent time, and/or (depending on its dissolve time) at a different section of gastrointestinal tract.

In one embodiment, the multi-shell capsule comprises at least two active ingredients.

In another preferred embodiment of this invention, the multi-shell capsule comprises one or more primary shell(s) containing as an active ingredient and one or more secondary shell(s) containing an active ingredient, compressed with or to the primary shell(s), to form the multi-shell capsule.

In another preferred embodiment of this invention, the multi-shell capsule may further comprise an optional inert shell or shells to control release of the active ingredient from the secondary shell(s).

In another preferred embodiment of this invention, the multi-shell capsule may further comprise an optional functional or nonfunctional coating either partly or completely surrounding the shell.

In another preferred embodiment of this invention, the multi-shell capsule may further comprise an optional subcoating between the multi-shell capsule and the functional or nonfunctional coating.

Another aspect of the present invention relates to the secondary shell comprising a plurality of one single shell capsule for releasing at least one active ingredient.

Another aspect of the present invention relates to methods for treating a subject with the multi-shell capsule of the present invention.

### DETAILED DESCRIPTION OF DRAWINGS

The present invention relates to the multi-shell capsule for oral administration of a combination of active ingredients. The capsule is formulated so that the different shells release the active ingredients at gastrointestinal tract at different dissolvent time.

Any active ingredient administered orally can be formulated in accordance with the present invention as these multi-shell capsules provide a particularly useful formulation to achieve simultaneous release of two or more different active ingredients at different dissolvent time.

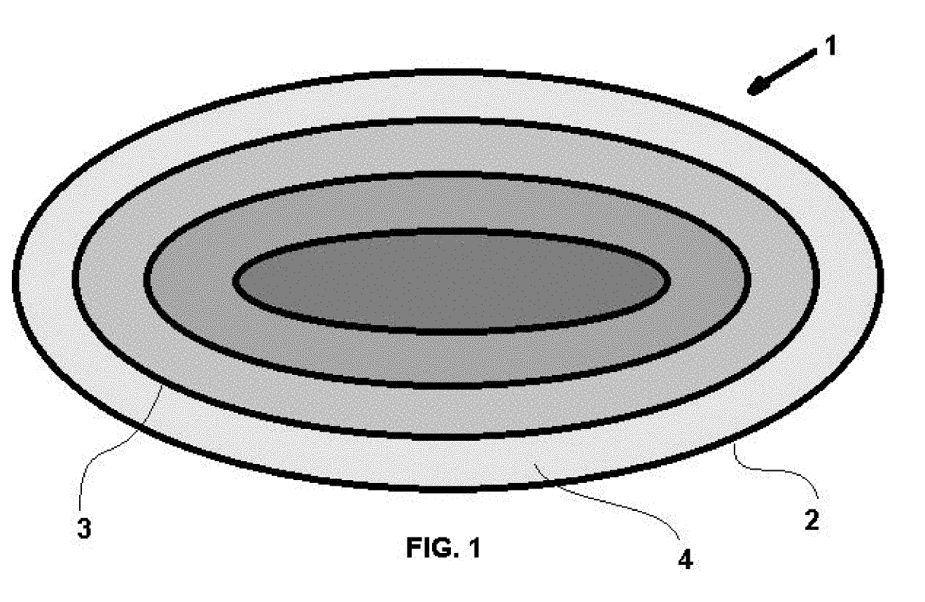

As depicted in FIG. 1, the multi-shell capsule comprises a multi-shell capsule 1 with a primary shell 2 containing an active ingredient and one or more secondary shell(s) 3 containing one or more active ingredient(s). The multi-shell capsule may further comprise an optional inert layer 4 in between the primary shell 2 and the secondary shell(s) 3 to control release of the active ingredient from the secondary shell(s).

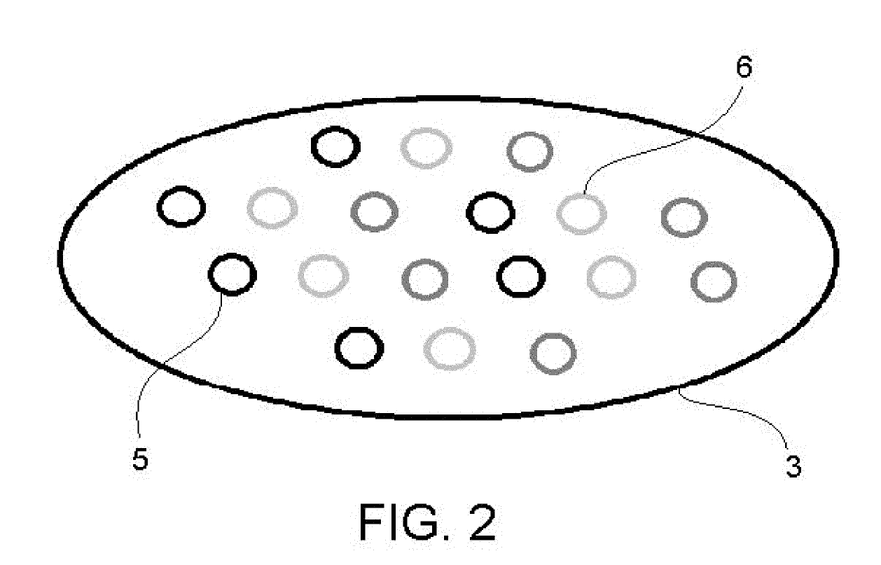

FIG. 2 illustrates that the multi-shell capsule 1 may further comprise a functional coating 5 and/or a non-functional coating 6

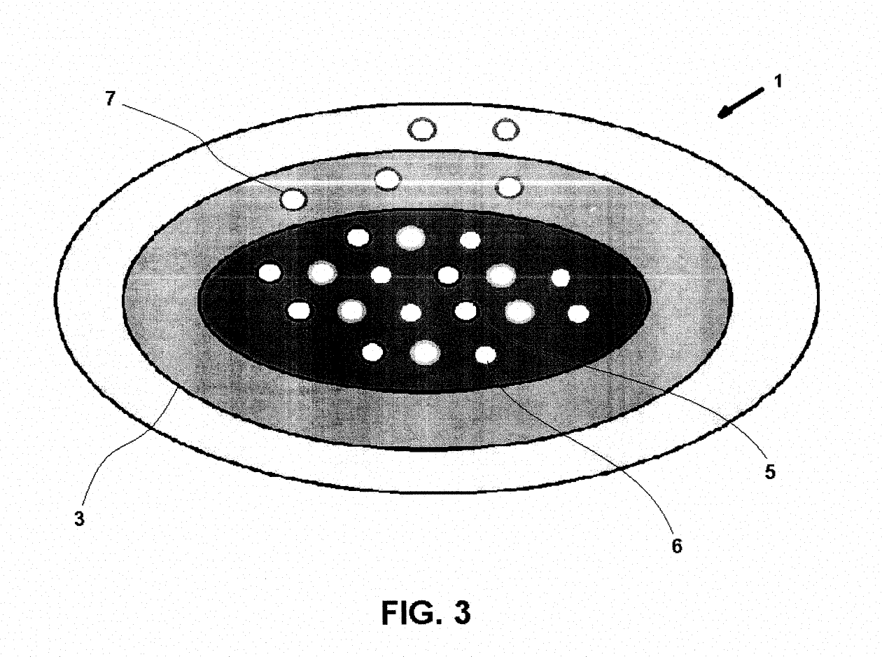

FIG. 3 shows an optional subcoating 7 in between the secondary shell(s) 3 and the functional coating 5 or non-functional coating 6.

Invention and its practical application to persons who are skilled in the art. As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above described exemplary embodiments, but should be defined only in accordance with the following claims appended hereto and their equivalents.

Having illustrated and described the principles of the present invention in a preferred embodiment, it will be apparent to those skilled in the art that the embodiment can be modified in arrangement and detail without departing from such principles. Any and all such embodiments are intended to be included within the scope of the following claims.

What is claimed is:

1. A multi-shell capsule comprising:
a primary shell having a different dissolving time for releasing a first active ingredient or set of ingredients; and at least one secondary shell having a different dissolving time for releasing a second active ingredient or set of ingredients, wherein said at least primary shell and said at least one secondary shell are compressed to form the single multi-shell capsule.

2. The multi-shell capsule of claim 1 wherein said at least secondary shell immediately dissolves subsequently to the dissolution of said primary capsule shell.

3. The multi-shell capsule of claim 2 further comprising one or more inert shells.

4. The multi-shell capsule of claim 3 further comprising at least one functional coating that either partly or completely surrounds said multi-shell capsule.

5. The multi-shell capsule of claim 4 wherein said at least functional coating permits quick release of the first active ingredient or set of ingredients from said primary shell while providing controlled release time of the second active ingredient or set of ingredients from said at least secondary shell.

6. The multi-shell capsule of claim 5 wherein said at least secondary shell further comprising at least one single shell capsule for releasing at least one active ingredient or set of ingredients.

7. The multi-shell capsule of claim 6 further comprising a subcoating between said at least secondary shell and said at least one single shell capsule.
